(19) United States
(12) Patent Application Publication
(10) Pub. No.: US 2024/0385101 A1
(43) Pub. Date: Nov. 21, 2024

(54) SYSTEMS AND METHODS FOR DETERMINING IN SITU CAPILLARY PRESSURE IN POROUS MEDIA

(71) Applicants: TOTALENERGIES E&P RESEARCH & TECHNOLOGY USA, LLC, Houston, TX (US); WILLIAM MARSH RICE UNIVERSITY, Houston, TX (US)

(72) Inventors: Sibani L. Biswal, Bellaire, TX (US); George J. Hirasaki, Bellaire, TX (US); Eric D. Vavra, Sugar Land, TX (US); Maura C. Puerto, Houston, TX (US); Khalid Mateen, Katy, TX (US); Kun Ma, Sugar Land, TX (US)

(73) Assignees: TOTALENERGIES E&P RESEARCH & TECHNOLOGY USA, LLC, Houston, TX (US); WILLIAM MARSH RICE UNIVERSITY, Houston, TX (US)

(21) Appl. No.: 18/696,191

(22) PCT Filed: Sep. 28, 2022
(86) PCT No.: PCT/US2022/045102

§ 371 Date: Mar. 27, 2024

Related U.S. Application Data
(60) Provisional application No. 63/250,848, filed on Sep. 30, 2021.

(51) Int. Cl. G01N 15/08; G01N 33/24

(52) U.S. Cl. CPC G01N 15/0826; G01N 33/24

(57) ABSTRACT

A capillary pressure assembly (50) for determining in situ capillary pressure in porous media (5) includes a hydrophobic member (170) including one or more ports (180) configured to communicate a pressure of a first phase of a fluid of the porous media to a first passage (103) of the capillary pressure assembly, wherein the porous media includes a consolidated porous media, a hydrophilic member (140) including a porous material configured to communicate a pressure of a second phase, immiscible with respect to the first phase, of the fluid of the porous media to a second passage (113) of the capillary pressure assembly, and a pressure sensor (60) configured to determine the capillary pressure in the porous media based on a pressure within the first passage and a pressure within the second passage.

### CROSS-REFERENCE TO RELATED APPLICATIONS

This application claims benefit of U.S. provisional patent application Ser. No. 63/250,848 filed Sep. 30, 2021, and entitled “Systems and Methods for Determining In Situ Capillary Pressure In Porous Media,” which is hereby incorporated herein by reference in its entirety for all purposes.

### STATEMENT REGARDING FEDERALLY SPONSORED RESEARCH OR DEVELOPMENT

Not applicable.

### BACKGROUND

In many, varying applications it may be desirable to determine properties of fluid(s) contained within porous media. As an example, processes within the oil and gas industry such as enhanced oil recovery (EOR), fluid diversion, gas sequestration, and matrix acidization may involve estimating various properties of fluids contained within porous media including, for example, subterranean earthen formations comprising oil-reservoir rocks. A property of particular interest in these processes is the capillary pressure of fluids contained in the porous medium. In general, capillary pressure refers to the difference in pressures between two immiscible phases of a fluid (flowing or stationary) that arises from surface or interfacial tension and a mean curvature of the fluid-fluid interface. For a given saturation, or fraction of the pore space of the porous medium occupied by one of the fluids, there exists an associated capillary pressure. The capillary pressure of fluids in a given porous medium may be used to provide useful insight regarding the suitability of EOR techniques or other processes.

### SUMMARY

An embodiment of a capillary pressure assembly for determining in situ capillary pressure in porous media comprises a hydrophobic member comprising one or more ports configured to communicate a pressure of a first phase of a fluid of the porous media to a first passage of the capillary pressure assembly, wherein the porous media comprises a consolidated porous media, a hydrophilic member comprising a porous material configured to communicate a pressure of a second phase, immiscible with respect to the first phase, of the fluid of the porous media to a second passage of the capillary pressure assembly, and a pressure sensor configured to determine the capillary pressure in the porous media based on a pressure within the first passage and a pressure within the second passage. In some embodiments, the hydrophilic member comprises a frit comprised of a porous material. In some embodiments, the frit is connected to a tube defining the second passage by a joint, and wherein an interface formed between the joint and the frit is sealed by a resin. In certain embodiments, an interface formed between the tube and the joint is sealed by an annular seal comprising a polymeric material. In certain embodiments, the porous material of the frit comprises at least one of a ceramic and a metallic material. In some embodiments, the hydrophobic member comprises a cap having one or more radial ports formed therein for communicating the pressure of the first phase to the first passage. In some embodiments, the cap comprises a fluorocarbon material. In some embodiments, the cap is sealed to a tube defining the first passage by a resin. In certain embodiments, the capillary pressure assembly comprises a compressible wrap positioned about the hydrophilic member and configured to maintain capillary continuity between the hydrophilic member and the porous media.

An embodiment of a system for determining the capillary pressure in the porous media comprises a core holder containing the porous media, wherein the porous media comprises a core sample, a capillary pressure assembly, wherein the capillary pressure assembly extends into the core sample and connects to the core holder, and a pump configured to flow a fluid from a fluid source through the core sample.

An embodiment of a capillary pressure assembly for determining in situ capillary pressure in porous media comprises a hydrophobic member comprising one or more ports configured to communicate a pressure of a first phase of a fluid of the porous media to a first passage of the capillary pressure assembly, a hydrophilic member comprising a porous material configured to communicate a pressure of a second phase, immiscible with respect to the first phase, of the fluid of the porous media to a second passage of the capillary pressure assembly, a compressible wrap positioned about the hydrophilic member and configured to maintain capillary continuity between the hydrophilic member and the porous media, and a pressure sensor configured to determine the capillary pressure in the porous media based on a pressure within the first passage and a pressure within the second passage. In some embodiments, the porous media comprises a consolidated porous media. In some embodiments, the first passage is defined by a first tube of the capillary pressure assembly and the second passage is defined by a second tube of the capillary pressure assembly, and wherein the first tube and the second tube comprise at least one of a metallic material and a polymer. In some embodiments, the first passage is defined by a first tube of the capillary pressure assembly and the second passage is defined by a second tube of the capillary pressure assembly, and wherein the first tube is positioned concentrically within the second tube whereby the second passage comprises an annulus formed radially between the first tube and the second tube. In certain embodiments, the hydrophilic member comprises a frit comprised of a porous material. In certain embodiments, the hydrophobic member comprises a cap having one or more radial ports formed therein for communicating the pressure of the first phase to the first passage.

An embodiment of a method for determining in situ capillary pressure in porous media comprises (a) inserting a capillary pressure assembly into the porous media whereby a hydrophobic member of the capillary pressure assembly communicates a pressure of a first phase of a fluid of porous media to a first passage of the capillary pressure assembly, and a hydrophilic member of the capillary pressure assembly communicates a pressure of a second phase, immiscible with respect to the first phase, of the fluid of the porous media to a second passage of the capillary pressure assembly, and (b) determining the capillary pressure in the porous media based on a pressure within the first passage and a pressure within the second passage. In some embodiments, (a) comprises contacting a surface of the porous media with a compressible wrap of the capillary pressure assembly positioned about the hydrophilic member. In some embodiments, the method further comprises (c) pumping the fluid of the porous media by a pump through the porous media as the capillary pressure in the porous media is determined by the capillary pressure assembly.

Embodiments described herein comprise a combination of features and characteristics intended to address various shortcomings associated with certain prior devices, systems, and methods. The foregoing has outlined rather broadly the features and technical characteristics of the disclosed embodiments in order that the detailed description that follows may be better understood. The various characteristics and features described above, as well as others, will be readily apparent to those skilled in the art upon reading the following detailed description, and by referring to the accompanying drawings. It should be appreciated that the conception and the specific embodiments disclosed may be readily utilized as a basis for modifying or designing other structures for carrying out the same purposes as the disclosed embodiments. It should also be realized that such equivalent constructions do not depart from the spirit and scope of the principles disclosed herein.

### BRIEF DESCRIPTION OF THE DRAWINGS

For a detailed description of various exemplary embodiments, reference will now be made to the accompanying drawings in which:

FIG. 1 is a schematic view of an embodiment of a system for determining capillary pressure in porous media in accordance with the principles described herein;

FIG. 2 is a schematic view of another embodiment of a system for determining capillary pressure in porous media in accordance with the principles described herein;

FIG. 3 is a schematic view of another embodiment of a system for determining capillary pressure in porous media in accordance with the principles described herein;

FIG. 4 is a schematic view of another embodiment of a system for determining capillary pressure in porous media in accordance with the principles described herein;

FIG. 5 is an enlarged cross-sectional view of the capillary pressure probe of the system of FIG. 1;

FIG. 6 is a partial, enlarged, cross-sectional view of the hydrophobic member of the capillary pressure probe of FIG. 5; and

FIG. 7 is a flowchart illustrating an embodiment of a method for determining capillary pressure in porous media in situ in accordance with the principles described herein.

### DETAILED DESCRIPTION

The following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection of the two devices, or through an indirect connection that is established via other devices, components, nodes, and connections. In addition, as used herein, the terms “axial” and “axially” generally mean along or parallel to a particular axis (e.g., central axis of a body or a port), while the terms “radial” and “radially” generally mean perpendicular to a particular axis. For instance, an axial distance refers to a distance measured along or parallel to the axis, and a radial distance means a distance measured perpendicular to the axis. As used herein, the terms “approximately,” “about,” “substantially,” and the like mean within 10% (i.e., plus or minus 10%) of the recited value. Thus, for example, a recited angle of “about 80 degrees” refers to an angle ranging from 72 degrees to 88 degrees.

As described above, capillary pressure of fluids contained in porous media (e.g., core samples from subterranean earthen formations and/or other natural environments) is a parameter of interest in various applications including processes associated with the oil and gas industry such as, for example, EOR. Conventionally, capillary pressure in porous media is determined indirectly as a function of saturation with methods such as mercury injection capillary pressure (MICP), porous diaphragm, or centrifugation. These conventional, indirect strategies typically impose a capillary pressure upon a fluid and measure a corresponding saturation of the fluid. Conventional indirect strategies for determining capillary pressure of fluids in porous media usually require assumptions to be made regarding parameters of the porous media and fluids therein in calculating the capillary pressure given that these conventional indirect methods are not performed in situ. Such assumptions may undesirably hinder the accuracy of the calculated capillary pressure and prevent the making of in situ measurements. Moreover, may not provide a real-time determination of the capillary pressure in the porous media and may require additional, specialized equipment (e.g., centrifuges, specialized core holders, etc.).

Accordingly, embodiments disclosed herein include systems and methods for determining capillary pressure of fluids in porous media in situ. In other words, the systems and methods described herein allow for the direct measurement of capillary pressure in porous media by engaging and contacting a capillary pressure assembly with the porous media whereby a pressure sensor of the capillary pressure assembly can determine the capillary pressure in the porous media by determining a difference in pressure between a pair of immiscible phases of fluids in the porous media. The direct determination of the capillary pressure in the porous media offers the potential for a more accurate determination of the capillary pressure as compared to conventional indirect techniques that rely on various assumptions. As will be discussed further herein, capillary pressure assemblies described herein may include a capillary pressure probe including a hydrophilic member and a hydrophobic member configured to isolate the pressures of the distinct phases present in the porous media.

Embodiments of the systems described herein can also determine a capillary pressure in both consolidated and unconsolidated porous media and at elevated pressures and temperatures including, in at least some embodiments pressures up to 60,000 pounds per square inch (PSI) and temperatures in excess of 500 degrees fahrenheit (° F.). As used herein, the term “unconsolidated” porous media refers to media comprising grains which are separate from one another (e.g., sand) where “consolidated” porous media refers to media comprising grains which are chemically cemented together to form a rock (e.g., sandstone). Systems described herein may be utilized to directly determine capillary pressure in porous media comprising a consolidated material such as sandstone. As will be discussed further herein, embodiments of capillary pressure probes described herein may include a compressible wrap positioned about a hydrophilic member thereof configured to maintain capillary continuity between the hydrophilic member and the consolidated material comprising the porous media. The capillary pressure probe may also comprise materials and features configured to withstand corrosive fluids and elevated temperatures and pressures.

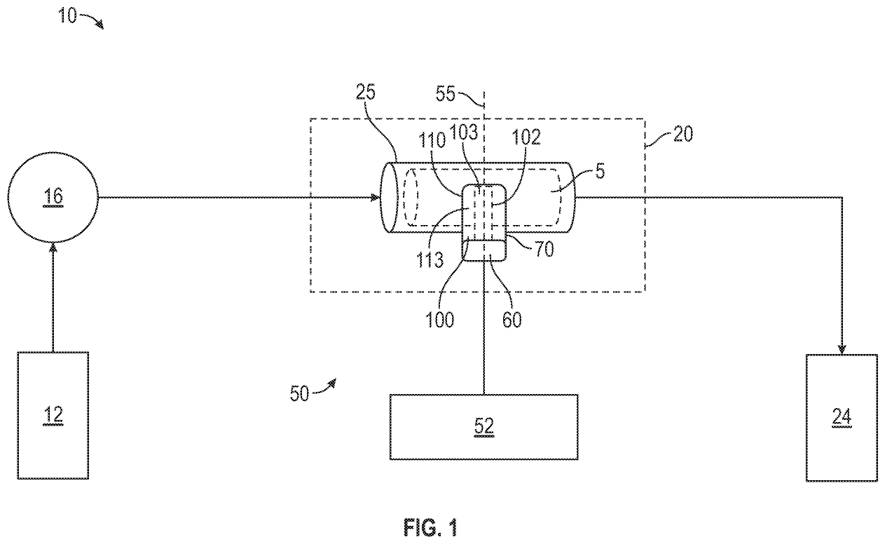

Referring now to FIG. 1, a system 10 for determining in situ capillary pressure in a porous medium 5 is shown. In this exemplary embodiment, porous medium 5 comprises a consolidated porous medium such as, for example, a consolidated rock (e.g., sandstone, etc.). Thus, porous medium 5 may also be referred to herein as core 5 or core sample 5. Porous medium 5 may be obtained from a natural environment (e.g., a subterranean earthen formation) of interest or may comprise an analog core sample intended to mimic porous media of the natural environment of interest. Additionally, in other embodiments, the porous medium 5 may comprise an unconsolidated porous medium such as, for example, sand and/or soil.

In this exemplary embodiment, system 10 generally includes a fluid source 12, a pump 16, a heating device 20, a core holder 25 housing the porous medium 5, a fluid return 24, and an electronics package 30. System 10 is only shown schematically in FIG. 1, and thus may include features not illustrated in FIG. 1. System 10 is generally configured to flow a test or sample fluid from the fluid source 12 through the porous medium 5 while monitoring one or more parameters, including a capillary pressure, of the porous medium 5 using the electronics package 30 of system 10. In some embodiments, fluid source 12 includes one or more separate accumulators storing separate fluids that are mixed to form the sample fluid prior to being delivered to the porous medium 5; however, in other embodiments, the configuration of fluid source 12 may vary. The sample fluid delivered to the porous medium 5 from fluid source 12 may comprise two or more immiscible phases (e.g., gas and liquid, etc.). Additionally, the sample fluid delivered to porous medium 5 may be the same as or analogous to the fluid contained in the natural environment of interest. Thus, the sample fluid delivered to porous medium 5 may comprise corrosive fluids including, for example, supercritical CO2.

Pump 16 of system 10 pumps the sample fluid from fluid source 12 into and through the porous medium 5 housed within core holder 25. In general, pump 16 may comprise any suitable device for producing a flow of fluid. After flowing through porous medium 5, the sample fluid is delivered to fluid return 24 of system 10. In some embodiments, the sample fluid, having flowed through porous medium 5, may eventually return to the fluid source 12. Additionally, various valves may be connected between fluid source 12, pump 16, and fluid return 24 of system 10 to control the flow of the sample fluid therebetween. In some embodiments, pump 16 may pressurize the sample fluid delivered to porous media 5 to a pressure, in at least some applications, exceeding 60,000 PSI. In this exemplary embodiment, core holder 25 and porous medium 5 are each positioned within heating device 20, thereby allowing heating device 20 to maintain porous medium 5 and/or the sample fluid delivered to porous medium 5 from fluid source 12 at a desired elevated temperature which may, in some embodiments, be in excess of 500° F. Heating device 20 may comprise a convection oven, but in general, heating device 20 may be any suitable device for controlling the temperature of porous medium 5 and/or the sample fluid delivered to porous medium 5. Additionally, system 10 may include a fluid pressure regulator located between core holder 25 and fluid return 24 to maintain a desired pressure of the sample fluid exiting the core holder 25.

In this exemplary embodiment, system 10 also includes a capillary pressure assembly 50 generally configured to directly determine or measure capillary pressure in porous medium 5 in situ pressed directly into or against the porous medium 5. As used herein, the term “in situ” describes a capillary pressure assembly (e.g., capillary pressure assembly 50) being pressed directly into or against porous media under desired fluid flow conditions. It may be understood that in some applications the sample fluid within the porous medium in which the capillary pressure assembly is pressed may be stationary. Thus, capillary pressure assembly 50 may also be referred to herein as an in situ capillary pressure sensor 50, where capillary pressure sensor 50 is configured to, in situ, determine capillary pressure in both consolidated (e.g., porous medium 5) and unconsolidated porous media. As will be described further herein, features of capillary pressure assembly 50 enable it to determine the capillary pressure of fluids (flowing or stationary) that are at high pressures and temperatures, including supercritical fluids

Referring still to FIG. 1, in this exemplary embodiment, capillary pressure assembly 50 has a central or longitudinal axis 55 and generally includes an electronics package 52, a pressure sensor 60, and a capillary pressure probe 100. Electronics package 52 of capillary pressure assembly 50 is in signal communication with pressure sensor 60. Electronics package 52 is generally configured to determine the capillary pressure of the sample fluid in the porous medium 5 based on measurements provided by pressure sensor 60. Electronics package 52 may include one or more processors and one or more memory devices in signal communication with the one or more processors whereby the one or more processors may execute instructions stored on the one or more memory devices. In some embodiments, the memory device of electronics package 52 stores measurements performed by the pressure sensor 60 to be read by personnel of system 10.

The pressure sensor 60 is coupled to the capillary pressure probe 100 and is configured to measure or monitor pressures of fluids disposed in the capillary pressure probe 100. In this exemplary embodiment, pressure sensor 60 includes a differential pressure sensor or transducer configured to measure or determine a difference in pressure between a gas and a liquid each disposed in the capillary pressure probe 100. Particularly, in this exemplary embodiment, capillary pressure probe 100 comprises an inner tube 102 and an outer tube 110. Inner tube 102 of capillary pressure probe 100 defines an inner or central passage 103. An annulus 113 is formed radially between the inner tube 102 and outer tube 110, and thus, the annulus 113 surrounds the inner tube 102. In this exemplary embodiment, capillary pressure probe 100 has a central or longitudinal axis that is coaxially aligned with central axis 55 of capillary pressure assembly 50. Thus, central axis 55 may also be referred to herein as the central axis 55 of capillary pressure probe 100.

In this exemplary embodiment, pressure sensor 60 measures or determines a difference in a pressure of gas present in the central passage 103 and a pressure of a liquid present in the annulus 113. The gas and liquid are each in fluid communication with corresponding gas and aqueous phases of the sample fluid within porous medium 5. This difference in pressure between the gas in central passage 103 and the liquid in annulus 113 can be communicated to electronics package 52 for storage in a memory device thereof or indicated in real-time to personnel of system 10. In this manner, the capillary pressure of the sample fluid in porous medium 5 can be determined by capillary pressure assembly 50 and indicated to personnel of system 10 in real-time.

In addition to the components described above, capillary pressure assembly 50 includes an annular connector 70 which sealably connects to the core holder 25 to thereby provide a sealed connection between the capillary pressure assembly 50 and the core holder 25. In some embodiments, connector 70 may form a metal-to-metal seal with the core holder 25; however, in other embodiments, capillary pressure assembly 50 may not include connector 70.

In an exemplary embodiment, the central passage 103 of capillary pressure probe 100 is filled with a first single phase fluid and the annulus 113 may be filled with a second single phase fluid (of a different phase and immiscible with the first phase) prior to pressing the capillary pressure assembly 50 into porous medium 5. For example, central passage 103 may be pre-filled with an aqueous phase of the sample fluid delivered from fluid source 12 while the annulus 113 may be pre-filled with the gas phase of the sample fluid delivered from fluid source 12. In other embodiments, the phases contained in annulus 113 and central passage 103 may vary. For example, in some embodiments, the central passage 103 may be filled with a nonpolar liquid miscible with the aqueous phase of the sample fluid delivered from fluid source 12. In other embodiments, central passage 103 may be filled with the gas phase while annulus 113 is filled with the aqueous phase.

With the central passage 103 and annulus 113 filled with the respective phases of the sample fluid delivered from fluid source 12, pump 16 of system 10 may be operated to pump the sample fluid from fluid source 12 through the porous medium 5 stored within core holder 25. With capillary pressure probe 100 in contact with porous medium 5, the pressure sensor 60 of capillary pressure assembly 50 determines the capillary pressure of the sample fluid flowing through porous medium 5, which may then be indicated or communicated to operators of system 10.

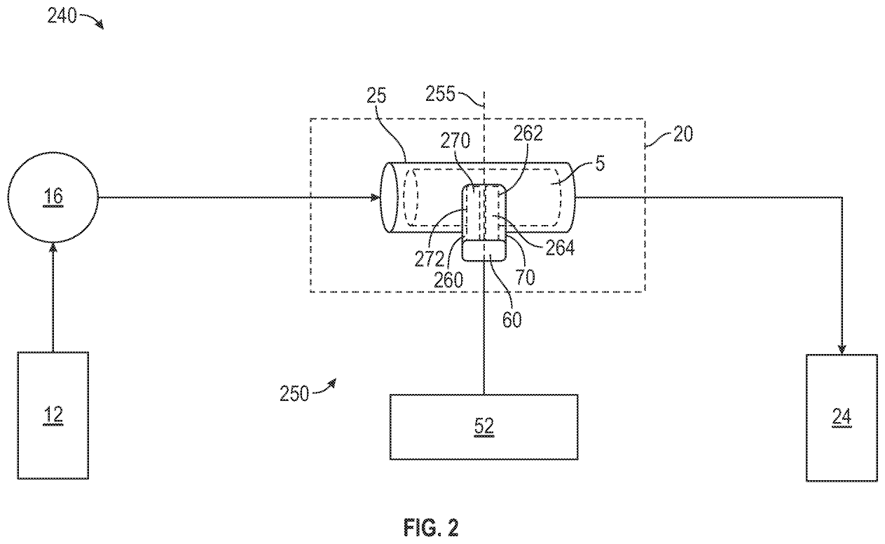

In this exemplary embodiment, the inner passage 103 and annulus 113 are positioned concentrically and coaxially aligned with respect to the central axis 55 of capillary pressure probe 100. However, in other embodiments, inner passage 103 and annulus 113 may not be concentric and coaxially aligned with respect to central axis 55. For example, referring briefly to FIG. 2, another embodiment of a system 240 for determining in situ capillary pressure in porous medium 5 is shown. In this exemplary embodiment, system 240 includes a downhole or capillary pressure assembly 250 that comprises a capillary pressure probe 260 having a central or longitudinal axis 255. In addition, the capillary pressure probe 260 includes a first tube 262 and a second tube 270 laterally offset from first tube 262. Particularly, in this exemplary embodiment, each tube 262, 270 is radially or laterally offset from the central axis 255 of capillary pressure probe 260. However, in other embodiments, at least one of the tubes 262, 270 may be aligned with central axis 255. Additionally, tubes 262, 270 of capillary pressure probe are not positioned concentrically. Similar to capillary pressure probe 100, a first passage 264 defined by the first tube 262 of capillary pressure probe 260 is filled with a first single phase fluid and a second passage 272 defined by second tube 270 is filled with a second single phase fluid (of a different phase and immiscible with the first phase) prior to the insertion of capillary pressure probe 260 into porous medium 5. The capillary pressure of the sample fluid flowing through porous medium 5 may be determined based on the pressures within passages 264, 272 by the pressure sensor 60 of capillary pressure assembly 250.

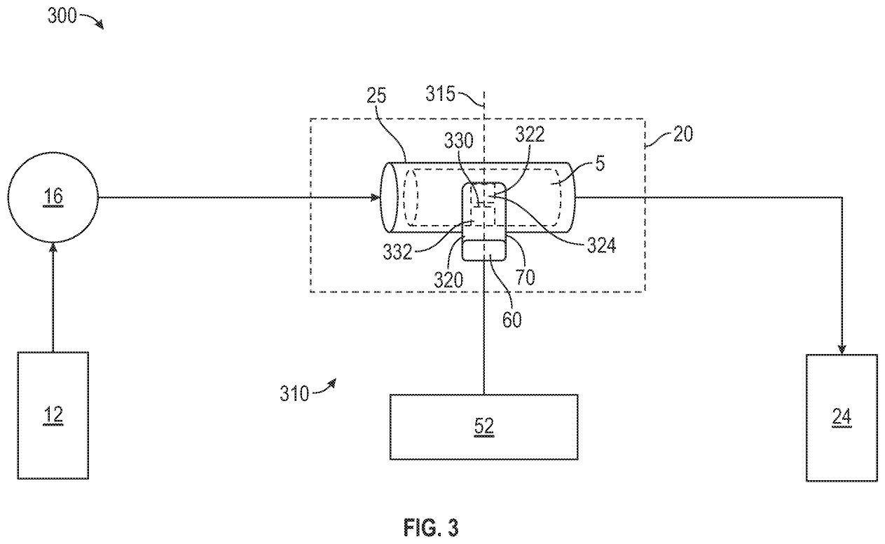

Referring briefly now to FIG. 3, yet another embodiment of a system 300 for determining in situ capillary pressure in porous medium 5 is shown. In this exemplary embodiment, system 300 includes a downhole or capillary pressure assembly 310 that comprises a capillary pressure probe 320 having a central or longitudinal axis 315. In addition, the capillary pressure probe 320 includes a first tube 322 and a second tube 330 that is longitudinally spaced (relative to central axis 315) from the first tube 322. In this exemplary embodiment, tubes 322, 330 are positioned along the central axis 315 but longitudinally spaced from each other along axis 315. Similar to capillary pressure probes 100, 260, a first passage 324 defined by the first tube 322 of capillary pressure probe 320 is filled with a first single phase fluid and a second passage 332 defined by second tube 330 is filled with a second single phase fluid (of a different phase and immiscible with the first phase) prior to the deployment of capillary pressure assembly 310 into borehole 344. The capillary pressure of the sample fluid flowing within porous medium 5 may be determined based on the pressures within passages 324, 332 by the pressure sensor 60 of capillary pressure assembly 310.

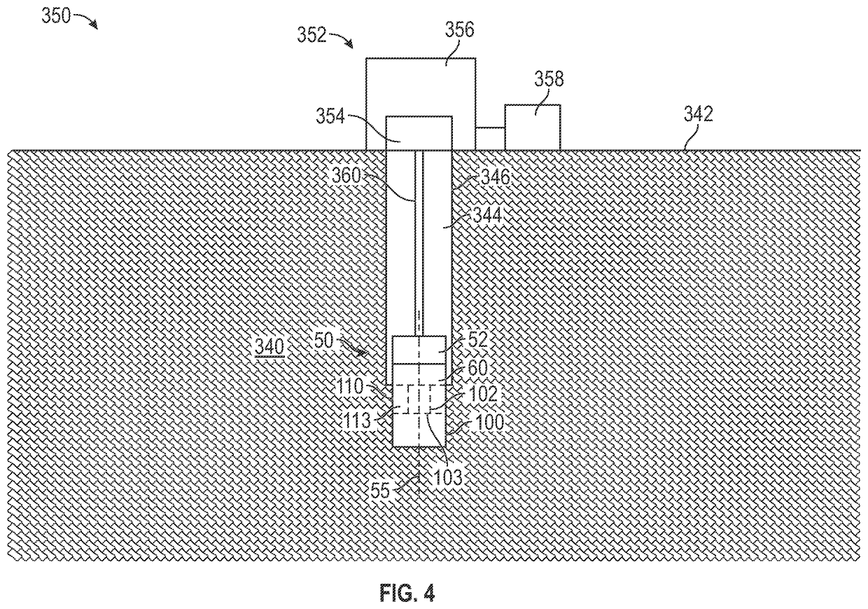

Referring now to FIG. 4, another embodiment of system 350 for determining in situ capillary pressure in porous media 340 is shown. Unlike systems 10, 240, and 300 described above, system 350 shown in FIG. 4 allows for the capillary pressure assembly 50 to be deployed directly into the natural environment of interest rather than relying on a sample taken from or designed to mimic the natural environment of interest.

System 350 may be utilized for determining capillary pressure in both consolidated porous media and unconsolidated porous media. In the exemplary embodiment shown in FIG. 4, porous media 340 comprises a consolidated porous media, and more specifically, is a consolidated subterranean earthen formation. Accordingly, porous media 340 may also be referred to herein as earthen formation 340. However, in other applications, system 350 may similarly be used to determine capillary pressure in unconsolidated porous media. Further, system 350 may be used to measure the capillary pressure of fluids, including supercritical fluids (e.g., supercritical carbon dioxide) at relatively high pressures and temperatures in the earthen formation 340 including, in at least some applications, pressures up to 60,000 PSI and temperatures in excess of 500° F.

In this embodiment, system 350 generally includes a surface assembly 352 positioned at the surface 342 and the capillary pressure assembly 50 which is positionable within a borehole 344 extending from the surface 342 through the earthen formation 340. Borehole 344 may be pre-drilled by a drilling system not shown in FIG. 4. In other embodiments, capillary pressure assembly 50 can be conveyed into and through borehole 344 via the drilling system used for form borehole 344 in lieu of the surface assembly 352 shown in FIG. 4.

Surface assembly 352 is configured to deploy capillary pressure assembly 50 into and from the borehole 344. Surface assembly 352 is configured for communicating with capillary pressure assembly 50 when capillary pressure assembly 50 is positioned within borehole 344 (i.e., downhole). In this exemplary embodiment, surface assembly 352 generally includes a containment system 354, a conveyance system 356, and a communication system 358. Containment system 354 includes one or more valves or other fluid containment devices configured to selectably seal borehole 344 from the ambient environment at the surface 342. In this manner, fluid communication between the borehole 344 and the surface ambient environment can be selectively isolated or restricted through the operation of containment system 354 of surface assembly 352.

The conveyance system 356 of surface assembly 352 can be operated to extend capillary pressure assembly 50 into and through borehole 344, and to retract capillary pressure assembly 50 from borehole 344. In this exemplary embodiment, conveyance system 356 includes a wireline injection system configured to inject a wireline 360 coupled to the capillary pressure assembly 50 at a terminal end thereof. Wireline 360 can be unwound from a surface reel of the conveyance system 356. Additionally, wireline 360 includes a signal conductor such as, for example, an electrical cable providing signal connectivity between the capillary pressure assembly 50 and the conveyance system 356. In other embodiments, the configuration of conveyance system 356 may vary. For example, in some embodiments, conveyance system 356 includes a system configured to inject a slickline into the borehole 344, with the capillary pressure assembly 50 being coupled to a terminal end of the slickline. In still other embodiments, conveyance system 356 includes a winch system configured to lower capillary pressure assembly 50 through borehole 344 via steel cable from which the capillary pressure assembly 50 is suspended. In still other embodiments, conveyance system 356 includes a coiled tubing injector configured to run coiled tubing or another tubular assembly into and from the borehole 344 with the capillary pressure assembly 50 coupled to a terminal end of the coiled tubing.

Communication system 358 is configured to communicate signals and/or data with capillary pressure assembly 50 whereby measurements performed by capillary pressure assembly 50 are communicated to the surface 342 in real-time to allow the measurements to be accessed by operators of the system 350. In this manner, the amount of time required to obtain the measurements performed by the capillary pressure assembly 50 may be minimized such that the capillary pressure in earthen formation 340 can be determined as efficiently and quickly as possible.

As described above, in this exemplary embodiment, capillary pressure assembly 50 is placed in signal communication with the surface assembly 352 via the wireline 360 from which capillary pressure assembly 50 is suspended. Communication system 358 is connected to conveyance system 356 whereby communication system 358 is placed in signal communication with capillary pressure assembly 50. However, in other embodiments, communication system 358 may communicate with capillary pressure assembly 50 in alternative ways. For example, in some embodiments, communication system 358 may comprise a wireless transceiver configured to communicate wirelessly (e.g., electromagnetically, acoustically, via pressure pulse modulation, etc.) with a corresponding wireless transceiver of capillary pressure assembly 50. In still other embodiments, surface assembly 352 may not include communication system 358 and instead measurements performed by capillary pressure assembly 50 may be stored in a memory device of the capillary pressure assembly 50 that may be accessed once capillary pressure assembly 50 has been retrieved to the surface 342 by the conveyance system 356 of surface assembly 352.

In this exemplary embodiment, capillary pressure assembly 50 is generally configured to directly determine or measure capillary pressure in earthen formation 340 in situ while the capillary pressure assembly 50 is located within borehole 344. As described above, in this exemplary embodiment, capillary pressure assembly 50 is suspended from wireline 360 within borehole 344. However, capillary pressure assembly 50 may be conveyed into and from borehole 344 via a variety of conveyance mechanisms. Additionally, in at least some applications, capillary pressure assembly 50 may be utilized in laboratory testing that is not performed in situ.

Referring still to FIG. 4, in this exemplary embodiment, electronics package 52 of capillary pressure probe 100 is in signal communication with pressure sensor 60, and the communication system 358 of surface assembly 352 via wireline 360. Electronics package 52 may include a battery or other power source or may be powered by the surface assembly 352 via wireline 360. Electronics package 52 may include a port or electrical connector to allow it to connect to wireline 360. In some embodiments, electronics package 52 may comprise a wireless transceiver (e.g., an electronic transceiver, an acoustic transceiver, a pressure pulse modulator, etc.) to permit electronics package 52 to communicate wirelessly with the communication system 358 of surface assembly 352.

In this exemplary embodiment, following the filling of the central passage 103 and annulus 113 of capillary pressure probe 100 with the respective phases of the formation fluid of earthen formation 340, the capillary pressure assembly 50 may be deployed to a desired location within the borehole 344 via the conveyance system 356 of surface assembly 352. At the desired location within borehole 344, an outer surface of the capillary pressure probe 100 is pressed against a wall 346 of borehole 344 (the wall comprising the porous media forming earthen formation 340) to ensure contact between the capillary pressure probe 100 and the earthen formation 340. With capillary pressure probe 100 in contact with earthen formation 340, the pressure sensor 60 of capillary pressure assembly 50 determines the capillary pressure of the formation fluid of earthen formation 340, which may then be indicated or communicated to operators of system 350 via the signal connectivity provided between electronics package 52 of capillary pressure assembly 50 and the communication system 358 of surface assembly 352. Following the determination of the capillary pressure of earthen formation 340 by the capillary pressure assembly 50, the capillary pressure assembly 50 is retrieved to the surface 342.

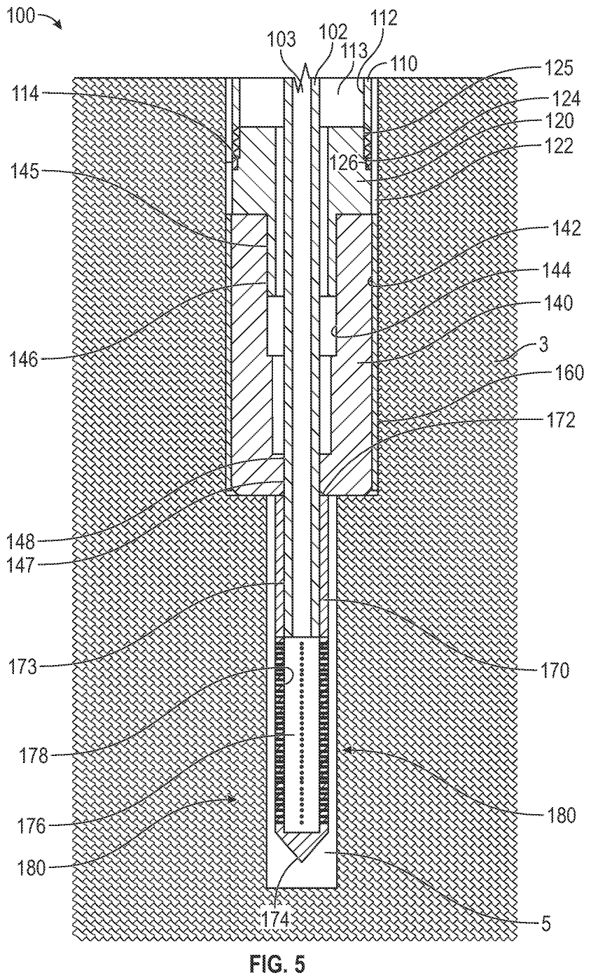

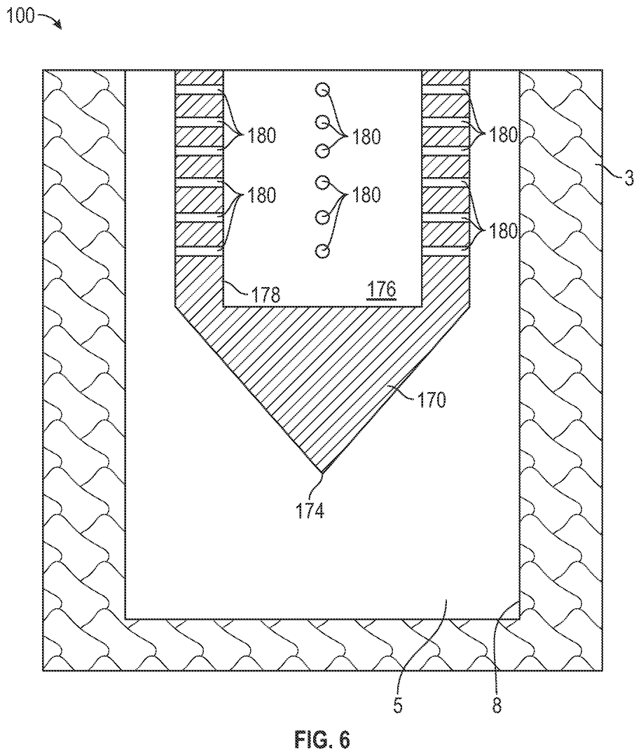

Referring now to FIGS. 5 and 6, the capillary pressure probe 100 of the capillary pressure assembly 50 shown in FIG. 1 is shown. In this exemplary embodiment, capillary pressure probe 100 includes the inner tube 102, the outer tube 110, an annular joint or coupler 120, an annular hydrophilic member or frit 140, and a hydrophobic member or cap 170. As will be further described herein, hydrophilic frit 140 is configured to allow the aqueous phase of a fluid within the porous media (i.e., a fluid of the porous media) to communicate with annulus 113 while restricting and/or preventing the gas phase of the fluid of the porous media from communicating with annulus 113 whereby the pressure within the annulus 113 assumes the pressure of the aqueous phase of the fluid of the porous media. Conversely, hydrophobic cap 170 is configured to allow the gas phase of fluid of the porous media to communicate with central passage 103 while restricting and/or preventing the aqueous phase of the fluid of the porous media from communicating with central passage 103 whereby the pressure within the central passage 103 assumes the pressure of the gas phase of the fluid of the porous media. The pressure of the gas phase of the fluid of the porous media varies from the pressure of the aqueous phase, thereby defining the capillary pressure of the fluid of the porous media.

The joint 120 of capillary pressure probe 100 couples the outer tube 110 to the hydrophilic frit 140 in a manner whereby an annular interface 125 between a generally cylindrical outer surface 122 of joint 120 and a generally cylindrical inner surface 112 of outer tube 110 is sealed from the borehole 344. In this exemplary embodiment, an annular seal or gasket 124 is positioned between an annular shoulder 126 formed on the outer surface 122 of joint 120 and a terminal end 114 of the outer tube 110, thereby sealing the interface 125. Gasket 124 is made of a material that is resistant to corrosion from the fluid of the porous media, as well as stable and suitable for use at the elevated temperatures and pressures present in the porous media. In this exemplary embodiment, the gasket 124 comprises a polymer such as Polytetrafluoroethylene (PTFE). However, in other embodiments, the configuration of gasket 124 may vary. In still other embodiments, sealing mechanisms other than gasket 124 may be utilized to seal the interface 125 between outer tube 110 and joint 120 such as metal-to-metal seals, resins, annular seal assemblies, or other mechanisms. Inner tube 102, outer tube 110, and joint 120 are each made of a durable, corrosion resistant material suitable for use in the porous media. For example, inner tube 102, outer tube 110, and joint 120 may be made of a metallic material such as for, example, a stainless-steel alloy, aluminum, Hastelloy®, Monel, etc. However, the material comprising inner tube 102, outer tube 110, and/or joint 120 may vary. For instance, in some embodiments, inner tube 102, outer tube 110, and/or joint 120 may comprise a polymer material such as, for example, Polyether ether ketone (PEEK).

Referring still to FIGS. 5 and 6, joint 120 is coupled to outer tube 110 whereby relative axial movement therebetween is restricted and/or prevented. In this exemplary embodiment, a releasable or threaded connector 116 is provided on the inner surface 112 and a corresponding releasable or threaded connector 128 is provided on the outer surface 122 of joint 120 whereby joint 120 releasably or threadably couples with outer tube 110. In other embodiments, mechanisms other than threads may be utilized to releasably couple joint 120 with outer tube 110. In other embodiments, outer tube 110 may permanently couple (e.g., via welding or other permanent joining techniques) with joint 120. In still other embodiments, the outer tube 110 may be formed integrally and monolithically with joint 120.

As described above, hydrophilic frit 140 allows for the pressure of the second phase (e.g., the aqueous phase) of the fluid of the porous media to be communicated to the annulus 113 of capillary pressure probe 100. Hydrophilic frit 140 comprises a porous material having a high capillary-entry pressure with respect to the first phase received in the central passage 103 of inner tube 102 (e.g., a gas phase, a nonpolar liquid phase, etc.). The capillary-entry pressure of the hydrophilic frit 140 with respect to the first phase may be higher than a maximum anticipated capillary pressure of the fluid of the porous media. In this manner, the first phase is inhibited from entering the annulus 113 through the hydrophilic frit 140. In this exemplary embodiment, the aqueous phase fluid in annulus 113 enters the pores of hydrophilic frit 140 and thereby wet the material forming the hydrophilic frit 140. The material forming the hydrophilic frit 140 is wetted by the fluid contained in annulus 113. As used herein, the term “hydrophilic material” is defined as a material having a water-in-air contact angle ranging between 0° to 90°. In some embodiments, the material comprising hydrophilic frit 140 may have a water-in-air contact angle approaching 0° to maximize the performance of frit 140.

In this exemplary embodiment, hydrophilic frit 140 comprises an annular cup including a generally cylindrical outer surface 142 extending between opposed longitudinal ends of the frit 140, and a generally cylindrical inner surface 144 also extending between the longitudinal ends of frit 140. Hydrophilic frit 140 is received over an end of the joint 120 whereby an annular interface 145 is formed between the inner surface 144 of frit 140 and the outer surface 122 of joint 120. In this exemplary embodiment, hydrophilic frit 140 is made of a ceramic material such as, for example, fired alumina, fired Talc, fired clay, and/or fired silica; however, in other embodiments, the material forming the hydrophilic frit 140 may be different. For example, in other embodiments, frit 140 may comprise a metallic material such as, for example, a stainless-steel alloy. In this exemplary embodiment, hydrophilic frit 140 is bonded to the joint 120 by a resin 146 (natural or synthetic) positioned along the annular interface 145 formed therebetween, thereby sealing interface 145. Additionally, a resin 148 is positioned along an annular interface 147 positioned between the inner surface 144 of hydrophilic frit 140 and a generally cylindrical outer surface 104 of the inner tube 102, thereby sealing the interface 147 formed therebetween.

In this exemplary embodiment, the resins 146, 148 each comprise an epoxy material; however, in other embodiments, the material used to seal interfaces 145, 147 may vary. In still other embodiments, mechanisms other than sealants such as resins may be utilized to seal interfaces 145, 147. For example, in embodiments where hydrophilic frit 140 is made of a metallic material, frit 140 may be threadably or otherwise releasably coupled to joint 120 at annular interface 145 with a gasket or other annular seal positioned along interface 145 to seal the connection formed between joint 120 and hydrophilic frit 140. Similarly, a gasket or other annular seal may be positioned along annular interface 148 to seal the interface 148 between frit 140 and inner tube 102.

In embodiments where capillary pressure probe 100 is utilized to determine a capillary pressure in a consolidated porous media, capillary pressure probe 100 may additionally include a compressible wrap 160 extending about the hydrophilic frit 140. Wrap 160 is configured to be compressed between the sidewall 8 of borehole 344 and the outer surface 142 of hydrophilic frit 140 whereby contact between sidewall 8 of borehole 344 and wrap 160 is maintained. Additionally, the wrap 160 is made of a hydrophilic material to maintain capillary continuity between the consolidated material (e.g., sandstone, etc.) and the hydrophilic frit 140. In this exemplary embodiment, wrap 160 comprises a cotton material; however, in other embodiments, other hydrophilic, compressible materials may be utilized such as, for example, hydrophilic cellulose-based absorbent materials (e.g. Kleenex® tissue, filter paper, and/or paper towel), hydrophilic sponges (e.g. polyacrylamide and/or polyurethane with open porosity), silica wool, and/or alumina wool.

As described above, hydrophobic cap 170 allows for pressure of the first phase (e.g., the gas phase) of the fluid of the porous media to be communicated to the annulus 113 of capillary pressure probe 100. In this exemplary embodiment, hydrophobic cap 170 comprises a hydrophobic material that is wetted by the fluid contained within the central passage 103 of inner tube 102. As used herein, the term “hydrophobic” material is defined as a material having a water-in-air contact angle between 90° and 180°. In some embodiments, the material comprising hydrophobic cap 170 may have a water-in-air contact angle approaching 180° to maximize the performance of hydrophobic cap 170.

Additionally, the material forming the hydrophobic cap 170 has a high capillary-entry pressure with respect to the second phase received in the annulus 113 (e.g., an aqueous phase, etc.). In this exemplary embodiment, hydrophobic cap 170 is made of a fluorocarbon material such as, for example, Polytetrafluoroethylene (PTFE). However, other hydrophobic materials may be utilized in other embodiments such as, for example any fluorocarbon polymer (e.g. fluorinated ethylene-propylene (FEP), polyethylenetetrafluoroethylene (ETFE), and/or Perfluoropolyether (PFPE)).

Hydrophobic cap 170 comprises a longitudinal first end 172, a longitudinal second end 174 opposite first end 172, and a central passage 176 defined by a generally cylindrical inner surface 178 that extends partially through hydrophobic cap 170 from the first end 172 thereof. In other words, central passage 176 does not extend entirely through hydrophobic cap 170 from first end 172 to second end 174, and instead terminates internally within cap 170. In this exemplary embodiment, hydrophobic cap 170 includes one or more radial ports 180 which extend entirely through cap 170 to thereby allow for the communication of pressure between the first phase of the fluid of the porous media and the fluid located within central passage 103. Each of the one or more radial ports 180 have a size or diameter small enough to prevent solid materials (e.g. sand, etc.) from passing therethrough. In some embodiments, the diameter of each radial port 180 ranges from approximately 10 micrometers (microns) to 100 microns; however, the diameter of each radial port 180 may vary. Additionally, in this exemplary embodiment, the first end 172 is heat shrunk onto an end of the inner tube 102 to thereby sealably couple the hydrophobic cap 170 to the inner tube 102 whereby fluid communication is restricted and/or prevented across an annular interface 173 formed therebetween. However, in other embodiments, hydrophobic cap 170 may be bonded (e.g., via a resin such as epoxy) or mechanically coupled to the inner tube 2 (e.g., via a releasable or threaded connector) which may be sealed by a gasket or other annular seal.

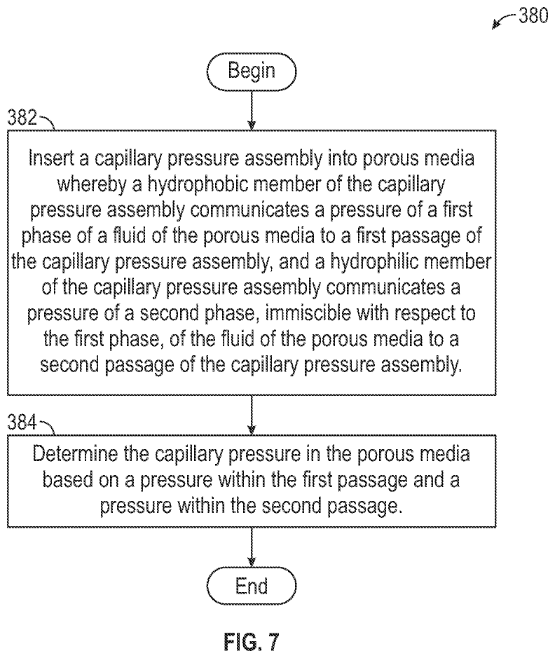

Referring now to FIG. 7, an embodiment of a method 380 for determining in situ capillary pressure in porous media is shown. Beginning at block 382, method 380 comprises inserting a capillary pressure assembly into the porous media. A hydrophobic member of the capillary pressure assembly communicates a pressure of a first phase of fluid of the porous media to a first passage of the capillary pressure assembly, and a hydrophilic member of the capillary pressure assembly communicates a pressure of a second phase, immiscible with respect to the first phase, of the fluid of the porous media to a second passage of the capillary pressure assembly. In some embodiments, block 382 comprises positioning the capillary pressure assembly 50 shown in FIG. 1 within porous media 5 whereby the hydrophobic cap 170 of the capillary pressure probe 100 shown in FIGS. 5 and 6 communicates a pressure of a first phase of fluid of the porous media to inner passage 103 while hydrophilic frit 140 of the capillary pressure probe 100 communicates a pressure of a second phase, immiscible with respect to the first phase, of the fluid of the porous media to the annulus 113.

At block 384, method 380 comprises determining the capillary pressure in the porous media based on a pressure within the first passage and a pressure within the second passage. In some embodiments, block 384 comprises determining the capillary pressure in the porous media 5 shown in FIG. 1 based on a pressure within the inner passage 103 of the capillary pressure probe 100 and a pressure within the annulus 113 of the capillary pressure probe 100. In some embodiments, block 384 comprises determining the capillary pressure based on a pressure within the first passage 264 of the capillary pressure assembly 250 shown in FIG. 2 and a pressure within the second passage 272 of the capillary pressure assembly 250. In certain embodiments, block 384 comprises determining the capillary pressure based on a pressure within the first passage 324 of the capillary pressure assembly 310 shown in FIG. 3 and a pressure within the second passage 332 of the capillary pressure assembly 310.

While exemplary embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

1. A capillary pressure assembly for determining in situ capillary pressure in porous media, the capillary pressure assembly comprising:
an annular hydrophobic member insertable into the porous media and comprising one or more ports configured to communicate a pressure of a first phase of a fluid of the porous media to a first passage of the capillary pressure assembly, wherein the porous media comprises a consolidated porous media;
an annular hydrophilic member insertable into the porous media and comprising a porous material configured to communicate a pressure of a second phase, immiscible with respect to the first phase, of the fluid of the porous media to a second passage of the capillary pressure assembly, wherein the hydrophilic member is coupled to the hydrophobic member along an annular interface formed between the hydrophobic member and the hydrophilic member; and
a pressure sensor configured to determine the capillary pressure in the porous media based on a pressure within the first passage and a pressure within the second passage.

2. The capillary pressure assembly of claim 1, wherein the hydrophilic member comprises a frit comprised of a porous material.

3. The capillary pressure assembly of claim 2, wherein the frit is connected to a tube defining the second passage by a joint, and wherein an interface formed between the joint and the frit is sealed by a resin.

4. The capillary pressure assembly of claim 3, wherein an interface formed between the tube and the joint is sealed by an annular seal comprising a polymeric material.

5. The capillary pressure assembly of claim 2, wherein the porous material of the frit comprises at least one of a ceramic and a metallic material.

6. The capillary pressure assembly of claim 1, wherein the hydrophobic member comprises a cap having one or more radial ports formed therein for communicating the pressure of the first phase to the first passage.

7. The capillary pressure assembly of claim 6, wherein the cap comprises a fluorocarbon material.

8. The capillary pressure assembly of claim 6, wherein the cap is sealed to a tube defining the first passage by a resin.

9. The capillary pressure assembly of claim 1, further comprising a compressible wrap positioned about the hydrophilic member and configured to maintain capillary continuity between the hydrophilic member and the porous media.

10. A system for determining the capillary pressure in the porous media, the system comprising:
a core holder containing the porous media, wherein the porous media comprises a core sample;
the capillary pressure assembly of claim 1, wherein the capillary pressure assembly extends into the core sample and connects to the core holder; and
a pump configured to flow a sample fluid from a fluid source through the core sample.

11. A capillary pressure assembly for determining in situ capillary pressure in porous media, the capillary pressure assembly comprising:
a hydrophobic member comprising one or more ports configured to communicate a pressure of a first phase of a fluid of the porous media to a first passage of the capillary pressure assembly;
a hydrophilic member comprising a porous material configured to communicate a pressure of a second phase, immiscible with respect to the first phase, of the fluid of the porous media to a second passage of the capillary pressure assembly;
a compressible wrap positioned about the hydrophilic member and configured to maintain capillary continuity between the hydrophilic member and the porous media; and
a pressure sensor configured to determine the capillary pressure in the porous media based on a pressure within the first passage and a pressure within the second passage.

12. The capillary pressure assembly of claim 11, wherein the porous media comprises a consolidated porous media.

13. The capillary pressure assembly of claim 11, wherein the first passage is defined by a first tube of the capillary pressure assembly and the second passage is defined by a second tube of the capillary pressure assembly, and wherein the first tube and the second tube comprise at least one of a metallic material and a polymer.

14. The capillary pressure assembly of claim 11, the first passage is defined by a first tube of the capillary pressure assembly and the second passage is defined by a second tube of the capillary pressure assembly, and wherein the first tube is positioned concentrically within the second tube whereby the second passage comprises an annulus formed radially between the first tube and the second tube.

15. The capillary pressure assembly of claim 11, wherein the hydrophilic member comprises a frit comprised of a porous material.

16. The capillary pressure assembly of claim 11, wherein the hydrophobic member comprises a cap having one or more radial ports formed therein for communicating the pressure of the first phase to the first passage.

17. A system for determining the capillary pressure in the porous media, the system comprising:
a core holder containing the porous media, wherein the porous media comprises a core sample;
the capillary pressure assembly of claim 11, wherein the capillary pressure assembly extends into the core sample and connects to the core holder; and
a pump configured to flow a sample fluid from a fluid source through the core sample.

18. A method for determining in situ capillary pressure in porous media, the method comprising:
(a) inserting a capillary pressure assembly into the porous media whereby a hydrophobic member of the capillary pressure assembly communicates a pressure of a first phase of a fluid of the porous media to a first passage of the capillary pressure assembly, and a hydrophilic member of the capillary pressure assembly communicates a pressure of a second phase, immiscible with respect to the first phase, of the fluid of the porous media to a second passage of the capillary pressure assembly; and
(b) determining the capillary pressure in the porous media based on a pressure within the first passage and a pressure within the second passage.

19. The method of claim 18, wherein (a) comprises contacting a surface of the porous media with a compressible wrap of the capillary pressure assembly positioned about the hydrophilic member.

20. The method of claim 18, further comprising:
(c) pumping the fluid of the porous media by a pump through the porous media as the capillary pressure in the porous media is determined by the capillary pressure assembly.
